(19) United States
(12) Patent Application Publication
(10) Pub. No.: US 2006/0241092 A1
(43) Pub. Date: Oct. 26, 2006

(54) Contraceptive regimens for lower-weight women

(76) Inventor: Freedolph D. Anderson, Virginia Beach, VA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/395,525

(22) Filed: Apr. 3, 2006

Related U.S. Application Data
(60) Provisional application No. 60/671,533, filed on Apr. 15, 2005.

(51) Int. Cl. A61K 31/56

(52) U.S. Cl. 514/170

(57) ABSTRACT

The present invention discloses methods of providing contraception to a female based on the body weight of the female. According to the methods provided, the body weight of the female is determined and a contraceptive regimen is selected based on the female's body weight. The selected contraceptive regimen is then administered to the female to provide contraception. In particular, the present invention discloses a method of providing contraception to a lower-weight female in which the body weight of the female is determined, and, if the body weight is less than about 150 pounds, a low-dose contraceptive regimen is selected and then administered to the female to provide contraception.

This application claims the benefit of the filing date of U.S. Appl. 60/671,533, filed Apr. 15, 2005, the contents of which are hereby incorporated by reference in its entirety.

### BACKGROUND OF THE INVENTION

1. Field of the Invention

The present invention relates to methods of female contraception based on body weight, particularly to methods of contraception for lower-weight women.

2. Background Art

The ovarian/menstrual cycle is a complex event characterized by an estrogen rich follicular phase and, after ovulation, a progesterone rich luteal phase. Each phase has a duration of approximately 14 days resulting in an intermenstrual interval of about 28 days. The endometrial tissue responds to the changes in hormonal milieu.

The onset of menstruation is generally considered to be the beginning of a new menstrual cycle and is generally counted as Day 1. During a span of about 5 to 7 days, the superficial layers of the endometrium, which grew and developed during the antecedent ovarian/menstrual cycle, are sloughed because demise of the corpus luteum in the non-fertile menstrual cycle is associated with a loss of progesterone secretion. Ovarian follicular maturation occurs progressively resulting in a rise in the circulating levels of estrogen, which in turn leads to new endometrial proliferation.

The dominant ovarian follicle undergoes ovulation at mid-cycle, generally between menstrual cycle days 12 to 16 and is converted from a predominantly estrogen source to a predominantly progesterone source (the corpus luteum). The increasing level of progesterone in the blood converts the proliferative endometrium to a secretory phase in which the tissue proliferation has promptly abated, leading to the formation of endometrial glands or organs. When the ovulated oocyte is viably fertilized and continues its progressive embryonic cleavage, the secretory endometrium and the conceptus can interact to bring about implantation (nidation), beginning about 6 to 8 days after fertilization.

If an ongoing pregnancy is to be established via implantation, the embryo will attach and burrow into the secretory endometrium and begin to produce human chorionic gonadotropin (HCG). The HCG in turn stimulates extended corpus luteum function, i.e., the progesterone production remains elevated, and menses do not occur in the fertile menstrual cycle. Pregnancy is then established.

In the non-fertile menstrual cycle, the waning level of progesterone in the blood causes the endometrial tissue to be sloughed. This starts a subsequent menstrual cycle.

Because endometrial proliferation serves to prepare the uterus for an impending pregnancy, manipulation of hormones and of the uterine environment can provide contraception. For example, estrogens are known to decrease follicle stimulating hormone (FSH) secretion by feedback inhibition. Under certain circumstances, estrogens can also inhibit luteinizing hormone (LH) secretion, once again by negative feedback. Under normal circumstances, the spike of circulating estrogen found just prior to ovulation induces the surge of gonadotropic hormones that occurs just prior to and resulting in ovulation. High doses of estrogen immediately post-coitally also can prevent conception probably due to interference with implantation.

Progestins can also provide contraception. Endogenous progesterone after estrogen is responsible for the progestational changes of the endometrium and the cyclic changes of cells and tissue in the cervix and the vagina. Administration of progestin makes the cervical mucus thick, tenacious and cellular, which is believed to impede spermatozoal transport. Administration of progestin also inhibits luteinizing hormone secretion and blocks ovulation in humans.

The most prevalent form of oral contraception is a pill that combines both an estrogen and a progestin, a so-called combined oral contraceptive preparation. Oral contraceptive preparations that contain progestin, without estrogen, are also available. These progestin-only contraceptives generally contain about one-third to one-fourth the dose of progestin present in the combined oral contraceptives. Despite their lower progestin doses, however, the progestin-only preparations have a more varied spectrum of side effects than do the combined preparations, especially more breakthrough bleeding. As a result, the combined preparations are the preferred oral contraceptives in use today (Sheth et al., Contraception 25:243 (1982)).

Several studies indicate that women in the highest weight quartiles (i.e., those weighing more than about 150 pounds) experience a slightly higher contraceptive failure rate than those in the lower two weight quartiles, particularly those higher-weight women using low-dose contraceptive regimens containing less than 30 μg of ethinyl estradiol or its equivalent. For example, in a recent study of body weight and oral contraceptive failure, women weighing about 70.5 kg or more were reported to have a 60% higher risk of oral contraceptive failure (Holt, V. L., et al., Obstet. Gynecol. 99:820-827 (2002)). See also Gu et al., Contraception 52:99-103 (1995), and Zieman, et al., Fertility and Sterility 77(Suppl. 2):S13-S18 (2002).

It is known that the estrogen component in many contraceptive regimens adds to contraceptive efficacy by inhibiting FSH stimulation of the ovarian follicle through feedback inhibition. One possible explanation for increased contraceptive failure in higher-weight women is that estrogen plasma levels, particularly during ovarian follicle maturation, are lower in higher-weight women using low-dose contraceptive regimens than in lower-weight women using the same low-dose regimen. This results in increased follicular activity in the higher-weight women that allows some follicles to mature enough to assume the state of a “dominant” follicle. Once a follicle in the ovary becomes “dominant,” control of further follicle growth becomes local rather than systemic, and the suppression of ovulation using contraceptive regimes becomes problematic. A similar situation can occur when contraceptive doses are missed in a regimen, especially at or around the time of the “pill-free interval” in an oral contraceptive regimen.

There exists a need for contraceptive regimens that are both safe and effective for women of all weights, in particular, contraceptive regimens that are both safe and effective for lower-weight women.

### BRIEF SUMMARY OF THE INVENTION

The present invention is directed to methods of providing contraception to a female based on the body weight of the female. According to the methods provided, the body weight of the female is determined and a contraceptive regimen is selected based on the female's body weight. The selected contraceptive regimen is then administered to the female to provide contraception. In particular, the present invention provides a method of providing contraception to a lower-weight female in which the body weight of the female is determined, and, if the body weight is less than about 150 pounds, a low-dose contraceptive regimen is selected and then administered to the female to provide contraception.

Thus, the present invention is directed to a method of providing contraception to a female in need thereof, the method comprising (a) determining a body weight of the female; (b) selecting a contraceptive regimen to administer to the female based on the body weight determined in (a); and (c) administering the contraceptive regimen selected in (b) to the female to provide contraception.

The present invention is also directed to a method of providing contraception to a lower-weight female in need thereof, the method comprising (a) determining a body weight of a female; (b) designating the female as a lower-weight female if the body weight determined in (a) is less than about 150 pounds; (c) selecting a low-dose contraceptive regimen to administer to the lower-weight female designated in (b); and (d) administering the low-dose contraceptive regimen in (c) to the lower-weight female to provide contraception.

In some aspects of the invention, (a) or (b) is performed by the female, or (c) or (d) is performed by the lower-weight female. In other aspects, (a), (b), (c), or (d) is performed by a licensed health care professional.

In some aspects of the invention, the female is designated as the lower-weight female if the body weight determined in (a) is less than about 130 pounds.

In some aspects, the low-dose contraceptive regimen comprises a combination of estrogen and progestin that is administered for a period of more than 20 consecutive days, in which the daily amount of estrogen is equivalent to about 5 μg to about 30 μg of ethinyl estradiol, and in which the daily amount of progestin is equivalent to about 10 μg about 150 μg of levonorgestrel. In other aspects, the daily amount of estrogen is equivalent to about 5 μg to about 25 μg of ethinyl estradiol, and the daily amount of progestin is equivalent to about 75 μg to about 125 μg of levonorgestrel; or the daily amount of estrogen is equivalent to about 10 μg to about 25 μg of ethinyl estradiol, and the daily amount of progestin is equivalent to about 75 μg to about 115 μg of levonorgestrel; or the daily amount of estrogen is equivalent to about 10 μg to about 20 μg of ethinyl estradiol, and the daily amount of progestin is equivalent to about 75 μg to about 100 μg of levonorgestrel. In yet other aspects, the daily amount of estrogen is equivalent to about 20 μg of ethinyl estradiol, and the daily amount of progestin is equivalent to about 100 μg of levonorgestrel.

In some aspects, the estrogen is ethinyl estradiol and the progestin is levonorgestrel.

In some aspects, the combination of estrogen and progestin is administered monophasically according to the method. In other aspects, the combination of estrogen and progestin is administered continuously.

In some aspects, the combination of estrogen and progestin is administered for a period of about 20 to about 30 consecutive days, or for a period of more than 50 consecutive days, or for a period of about 50 to about 110 consecutive days.

In some aspects of the invention, the contraceptive regimen further comprises a hormone-free period of about 2 to about 8 consecutive days, or about 2 to 5 consecutive days. In other aspects, the contraceptive regimen further comprises estrogen that is administered for a period of about 2 to about 10 consecutive days, in which the daily amount of estrogen is equivalent to about 5 μg to about 30 μg of ethinyl estradiol.

In some aspects, an antidepressant is administered according to the method (i) in combination with the estrogen that is administered for the period of about 2 to about 10 consecutive days, (ii) intermittently, (iii) one time, or (iv) once weekly.

In some aspects of the present invention, the combination of estrogen and progestin administered according to the method is for oral, vaginal, or transdermal administration.

In some aspects, the female is a peri-menopausal female.

The present invention is also directed to a method of providing contraception to a lower-weight female in need thereof, the method comprising (a) determining a body weight of a female; (b) designating the female as a lower-weight female if the body weight determined in (a) is less than about 150 pounds; and (c) administering monophasically to the lower-weight female a combination of estrogen and progestin for a period of more than 50 consecutive days, in which the daily amount of estrogen is equivalent to about 5 μg to about 30 μg of ethinyl estradiol, and in which the daily amount of progestin is equivalent to about 10 μg to about 150 μg of levonorgestrel, thereby providing contraception to the lower-weight female.

The present invention is further directed to a method of contraception for a lower-weight female in need thereof, the method comprising administering to the lower-weight female a combination of estrogen and progestin for a period of more than about 20 consecutive days in which the daily amount of estrogen is equivalent to about 5 μg to about 30 μg of ethinyl estradiol, and in which the daily amount of progestin is equivalent to about 10 μg to about 150 μg of levonorgestrel, wherein the combination of estrogen and progestin is administered only if the lower-weight female weighs less than about 130 pounds.

### DETAILED DESCRIPTION OF THE INVENTION

The present invention is directed to methods of providing contraception to a female based on the female's body weight. According to the methods provided herein, the body weight of the female is determined and a contraceptive regimen is selected based on the female's body weight. The selected contraceptive regimen is then administered to the female to provide contraception.

The selected contraceptive regimen can also be administered to the female to provide treatment for a menstrual-related condition or disorder, including, but not limited to, breakthrough bleeding, iron deficiency anemia, and menstrual disorders. Menstrual disorders include, but are not limited to, irregular cycles, dysmenorrhea (painful menstruation), mittelschmerz, and dysfunctional uterine bleeding, as well as premenstrual symptoms such as, but not limited to, those associated with premenstrual syndrome (PMS) or Premenstrual Dysphoric Disorder (PMDD). See, for example, Bell et al., U.S. Pub. No. US 2003/0139381 A1; Ben-Maimon et al., WO 04/098517; and Bell et al., WO 05/007112. The contents of each of these published patent applications are incorporated by reference herein in their entirety.

Thus, the present invention also provides a method of providing contraception to a female for the treatment of a condition or disorder, based on the female's body weight, wherein the body weight of the female is determined, a contraceptive regimen is selected based on the female's body weight, and the selected contraceptive regimen is administered to the female.

In particular, the present invention provides methods of providing contraception to a lower-weight female. In some aspects of the invention, the body weight of the female is determined and, if the body weight is less than about 150 pounds, a low-dose contraceptive regimen is selected and then administered to the female to provide contraception.

Thus, the present invention provides a method of providing contraception to a female in need thereof, the method comprising:

(a) determining a body weight of the female;

(b) selecting a contraceptive regimen to administer to the female based on the body weight determined in (a); and

(c) administering the contraceptive regimen selected in (b) to the female to provide contraception.

In the methods of the invention, the female's body weight is first determined. In some aspects of the invention, the female's body weight is determined directly by weighing the female. In other aspects, the body weight is determined by asking the female to provide her body weight, which was previously determined.

The female's body weight is then used to select a contraceptive regimen. In some aspects of the invention, if the female's body weight is less than 150 pounds, a low-dose contraceptive regimen is selected for administration to the female. In other aspects, if the female's body weight is less than about 140 pounds, or less than about 130 pounds, a low-dose contraceptive regimen is selected for administration to the female.

In some aspects of the invention, if the female's body weight is about 150 pounds or greater than about 150 pounds, a contraceptive regimen other than a low-dose contraceptive regimen is selected for administration to the female.

The contraceptive regimen selected for administration is any contraceptive regimen that prevents or reduces the likelihood of conception. Contraceptive regimens suitable for selection are discussed below and can be conventional or extended cycle regimens, including monophasic, biphasic, triphasic, or multiphasic regimens. Suitable contraceptive regimens are those in which one or more hormonal or non-hormonal active agents are administered. For example, suitable regimens are those in which one or more estrogens, one or more progestins, and/or a combination of an estrogen and progestin, are administered.

Once selected, the contraceptive regimen is then administered to the female. The contraceptive regimen can be administered by any route in which it is effective. For example, administration can be through oral, parenteral, subcutaneous, intravenous, intramuscular, intraperitoneal, transdermal, buccal, ocular, or vaginal routes, or by inhalation, by depot injections, or by hormone implants.

Pharmaceutical formulations containing the contraceptive regimen active agent(s) and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of the contraceptive regimen active agent(s) as taught herein.

Thus, in some aspects of the invention, the selected contraceptive regimen is administered orally, in a pill or tablet dosage form. In other aspects, it is administered vaginally in a vaginal ring or as a vaginal cream. Modes of administration of the selected contraceptive regimen are discussed in more detail below.

In some aspects of the invention, the contraceptive regimen selected according to the invention is administered to a female to provide contraception. In other aspects, the contraceptive regimen selected according to the invention is administered to a female to provide treatment for one or more menstrual-related conditions or disorders as described herein. The female can be of child-bearing age or a peri-menopausal female.

As used herein, the term “female” refers to a human female.

The term “peri-menopausal female” refers to a woman who has not yet definitely arrived at menopause but who is experiencing symptoms associated with menopause. “Peri-menopause” means “about or around the time of menopause.” It encompasses the years preceding the last menstrual period during which ovarian function declines and ultimately ceases and can include the presence of symptoms and irregular cycles.

The various aspects of the methods of the invention can be performed by one or more parties. The parties performing the various aspects of the methods of the invention can be the same or different. For example, one or more of the various aspects of the disclosed methods can by performed by the female receiving the contraceptive regimen or by a licensed health care professional. Examples of licensed health care professionals include, but are not limited to, a licensed physician, or a licensed practical nurse.

Thus, in some aspects of the invention, the body weight of the female is determined by the female by weighing herself. In other aspects, the female's body weight is determined by a licensed health care provider, such as, for example, by a licensed practical nurse who weighs the female during a medical examination. In some aspects of the invention, a licensed health care professional determines the body weight of the female and selects the contraceptive regimen to be administered. In other aspects, the female determines her own body weight and a licensed health care professional selects the contraceptive regimen. In yet other aspects, the female both determines her own body weight and selects the contraceptive regimen.

Contraceptive Regimens

The contraceptive regimen selected for administration is any contraceptive regimen that is effective in preventing or reducing the likelihood of conception. The selected contraceptive regimen can be a regimen in which one or more hormonal agents or one or more non-hormonal agents are administered as the active agent(s). Examples of hormonal agents include, but are not limited to, natural and synthetic estrogens and progestins. For example, the selected regimen can be one in which an estrogen is administered, or one in which a progestin is administered, or one in which a combination of an estrogen and a progestin is administered.

Suitable progestins for use in the present invention include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens. Prodrugs of suitable progestins can also be used in the extended cycle regimen of the present invention.

The expression “prodrug” denotes a derivative of a known direct acting drug, which derivative has enhanced delivery characteristics and therapeutic value as compared to the drug and is transformed into the active drug by an enzymatic or chemical process. Ethynodiol diacetate, which is converted in vivo to norethindrone, is an example of a progestin prodrug that can be used in the present invention. Additional examples of progestin prodrugs include, but are not limited to, norgestimate (which is converted in vivo to 17-deacetyl norgestimate, also known as norelgestromin), desogestrel (which is converted in vivo to 3-keto desogestrel, also known as etonogestrel), and norethindrone acetate (which is converted in vivo to norethindrone).

Suitable estrogens in the present invention include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens; and selective estrogen receptor modulators. Prodrugs of suitable estrogens can also be used in the extended cycle regimen of the present invention. Examples of estrogen prodrugs that can be used in the present invention include, but are not limited to, estradiol acetate (which is converted in vivo to 17β-estradiol) and mestranol (which is converted in vivo to ethinyl estradiol).

The contraceptive regimen selected according to the invention can be monophasic, biphasic, triphasic, or multiphasic. That is, the active agent(s) in the contraceptive regimen, e.g., the estrogen, the progestin, or the combination of estrogen and progestin, is administered to the female monophasically, biphasically, triphasically, or multiphasically.

As used herein, “monophasic” refers to the continuous use of one particular dose of active agent(s) of the contraceptive regimen during the period of administration of the active agent(s). “Biphasic” refers to administration of a first continuous dose of active agent(s) during a first portion of the period of administration of the active agent(s), with administration of a second continuous dose of active agent(s) during the second portion of the period of administration of the active agent(s). “Triphasic” refers to administration of first, second, and third continuous doses of active agent(s) during the first, second, and third portions, respectively, of the period of administration of the active agent(s). “Multiphasic” refers to administration of four or more continuous doses of active agent(s) during the first, second, third, and fourth or more portions, respectively, of the period of administration of the active agent(s).

The selected contraceptive regimen can also be a conventional cycle regimen or an extended cycle regimen. As used herein, the term “conventional cycle regimen” refers to a contraceptive regimen that is administered on the basis of a 28-day to 31-day cycle. An example of a conventional cycle regimen is one in which a combination of estrogen and progestin is administered for 21 to 26 consecutive days, e.g., for 22 to 25 consecutive days, 23 to 25 consecutive days, 25 consecutive days, 26 consecutive days, or 21 consecutive days. Administration of the combination of estrogen and progestin is then followed by a period of from 2 to 10 consecutive days, e.g., from 2 to 8 consecutive days, 3 to 7 consecutive days, or 7 days, in which no hormone is administered, for a total of 28 days per cycle. Alternatively, administration of the combination of estrogen and progestin is followed by a period of from 2 to 10 consecutive days, e.g., from 2 to 8 consecutive days, 3 to 7 consecutive days, or 7 days, in which estrogen without progestin (“unopposed estrogen”) is administered, for a total of 28 days per cycle.

Thus, for example, in some aspects of the invention, a conventional cycle regimen is selected in which a combination of estrogen and progestin is administered for about 20 to about 30 consecutive days, in which the daily amount of estrogen is equivalent to about 5 μg to about 35 μg of ethinyl estradiol and the daily amount of progestin is equivalent to about 0.01 mg to about 0.20 mg of levonorgestrel.

The term “extended cycle regimen” refers to a regimen that is administered on the basis of a cycle that is more than 28 days. For example, an extended cycle regimen is one in which a combination of estrogen and progestin is administered continuously for more than 50 consecutive days, e.g., for 60 to 110 consecutive days, 81 to 110 consecutive days, or 81 to 89 consecutive days. Administration of the combination of estrogen and progestin is then followed by a period of from 2 to 10 consecutive days, e.g., for 2 to 8 consecutive days, 3 to 7 consecutive days, or for about 7 days, in which estrogen without progestin (“unopposed estrogen”) is administered. Alternatively, administration of the combination of estrogen and progestin can be followed by continuous administration of estrogen for 2 to 10 consecutive days, e.g., for 2 to 8 consecutive days, 3 to 7 consecutive days, or for about 7 days. An example of an extended cycle regimen is one in which a combination of estrogen and progestin is administered continuously for 84 days, followed by continuous administration of estrogen for 7 days, for a total of 91 days per cycle. See also, for example, U.S. Pat. No. 5,898,032 (Hodgen), which discloses contraceptive regimens comprising administration of a combination of estrogen and progestin for 60-110 days per cycle. Another example of an extended cycle regimen is one in which estrogen is administered continuously.

For example, in some aspects of the invention, an extended cycle regimen is selected in which a combination of estrogen and progestin is administered for more than 50 consecutive days, in which the daily amount of estrogen is equivalent to about 5 μg to about 35 μg of ethinyl estradiol and the daily amount of progestin is equivalent to about 0.01 mg to about 0.20 mg of levonorgestrel.

The term “continuous” or “consecutive” in reference to “administration” means that the frequency of administration is at least once daily. Note, however, that the frequency of administration can be greater than once daily and still be “continuous,” e.g., twice or even three times daily, as long as the specified dosage levels are not exceeded.

The term “dosage level,” “daily amount,” or “daily dosage amount” in reference to a contraceptive regimen active agent(s), e.g., estrogen or progestin, means the total amount of that active agent(s) administered per day. Thus, for example, “continuous administration” of a progestin to a woman at a “dosage level” of 30 μg means that the woman receives a total of 30 μg of progestin on a daily basis, whether the progestin is administered as a single 30 μg dose or as multiple doses, e.g., three separate 10 μg doses. A conventional means of continuously administering a contraceptive active agent(s) is as a single daily oral dose at the prescribed dosage level.

In the selected extended cycle regimen, the daily amount of estrogen is equivalent to about 5 μg to about 35 μg of ethinyl estradiol. In some aspects, the daily amount of estrogen is equivalent to about 5 μg to about 30 μg of ethinyl estradiol. In yet other aspects, the daily amount of estrogen is equivalent to about 10 μg to about 30 μg of ethinyl estradiol, or about 15 μg to about 25 μg of ethinyl estradiol. In some aspects of the invention, the daily amount of estrogen in the selected extended cycle regimen is equivalent to about 30 μg of ethinyl estradiol, or equivalent to about 20 μg of ethinyl estradiol

In the selected extended cycle regimen, the daily amount of progestin is equivalent to about 0.01 mg to about 0.25 mg of levonorgestrel. In some aspects of the invention, the daily amount of progestin is equivalent to about 0.01 mg to about 0.20 mg of levonorgestrel. In other aspects of the invention, the daily amount of progestin is equivalent to about 0.05 mg to about 0.15 mg of levonorgestrel. In yet other aspects, the daily amount of progestin is equivalent to about 0.15 mg of levonorgestrel, or equivalent to about 0.1 mg of levonorgestrel.

In some aspects of the invention, the estrogen and progestin of the selected extended cycle regimen are ethinyl estradiol and levonorgestrel, respectively, although other useful estrogens and progestins can be employed. The weight ratio of estrogen and progestin can be about 1:0.2 to about 1:300. In some aspects of the invention, the weight ratio of estrogen and progestin is about 1:1 to about 1:50. In other aspects of the invention, the weight ratio of estrogen and progestin is about 1:1 to about 1:10. For example, the daily amount of ethinyl estradiol is about 10 μg to about 30 μg and the daily amount of levonorgestrel is about 0.05 mg to about 0.150 mg.

The values given above are for ethinyl estradiol and levonorgestrel, and if a different estrogen or progestin or other active agent is employed, an adjustment in the amount based on the relative potency or activity can be made. Correlations in potency among the various estrogens and among the various progestins are known. See, for example, EP 0 253 607, which is hereby incorporated in its entirety by reference. For example, in a contraceptive regimen, 30 μg of ethinyl estradiol is roughly equivalent to about 60 μg of mestranol or about 2,000 μg of 17β-estradiol. Similarly, 0.050 mg of levonorgestrel is roughly equivalent to about 0.175 mg of norethindrone acetate, about 0.050 mg of desogestrel, about 0.050 mg 3-ketodesogestrel, about 0.035 mg of gestodene, about 0.100 mg of norgestrel, or about 0.375 mg trimegestone. It should be understood that when norgestrel is used in place of levonorgestrel, its concentration is twice that of levonorgestrel. Norgestrel (dl-norgestrel) is a racemic mixture of optically active isomers, while levonorgestrel is one of the optically active isomers present in norgestrel.

Thus, for example, in some aspects of the invention, the progestin used is trimegestone. The daily dosage of trimegestone can be, but is not limited to, about 0.25 mg to about 2.0 mg, about 0.5 mg to about 1.5 mg, or about 0.75 mg to about 1.25 mg. For example, in some aspects of the invention, the daily dosage of trimegestone is about 0.25 mg to about 1.5 mg, or about 0.375 mg to about 1.1 mg. In other aspects, the daily dosage of trimegestone is about 0.75 mg, about 1.0 mg, or about 1.1 mg.

Equivalent concentrations of estrogens and progestins can be determined using either in vitro or in vivo assay methods. See, for example, Kuhl, H., Drugs 51(2):188-215 (1996); Philibert, D., et al., Gynecol. Endocrinol. 13:316-326 (1999); and Lundeen, S., et al., J. Steroid Biochem. Molec. Biol. 78:137-143 (2001), in which the relative potencies of various progestins are compared using both in vitro and in vivo test assays. See also, for example, Dickey, R. P., “Contraceptive Therapy,” OBG Management Supplement (October 2000), pp. 2-6. Each of these documents is hereby incorporated by reference in its entirety.

Various combinations of progestin and estrogen that have been used in oral contraceptives are shown in Table 1.

TABLE 1
Combinations of Progestin and Estrogen

| | Norethindrone | | | EE Equivalent | | |
|---|---|---|---|---|---|---|
| | Dose | Equivalent | | Dose | Dose | |
| Progestin | (mg) | Dose (mg) | Estrogen | (mg) | (mg) | P/E Ratio |
| Norethynodrel | 9.85 | 9.85 | Mestranol | 0.150 | 0.105 | 93.810 |
| | 5.00 | 5.00 | | 0.075 | 0.053 | 95.238 |
| | 2.50 | 2.50 | | 0.036 | 0.025 | 99.206 |
| | 2.50 | 2.50 | | 0.100 | 0.070 | 35.714 |
| Norethindrone | 10.00 | 10.00 | Mestranol | 0.060 | 0.042 | 238.095 |
| | 2.00 | 2.00 | | 0.100 | 0.070 | 28.571 |
| | 1.00 | 1.00 | | 0.050 | 0.035 | 28.571 |
| | 1.00 | 1.00 | | 0.080 | 0.056 | 17.857 |
| Norethindrone | 1.00 | 1.00 | Ethinyl | 0.050 | 0.050 | 20.000 |
| | 1.00 | 1.00 | estradiol | 0.035 | 0.035 | 28.571 |
| | | | (EE) | | | |
| | 0.50 | 0.50 | | 0.035 | 0.035 | 14.286 |
| | 0.40 | 0.40 | | 0.035 | 0.035 | 11.429 |
| Norethindrone | 2.50 | 2.50 | EE | 0.050 | 0.050 | 50.000 |
| acetate | 1.00 | 1.00 | | 0.050 | 0.050 | 20.000 |
| | 0.60 | 0.60 | | 0.030 | 0.030 | 20.000 |
| | 1.50 | 1.50 | | 0.030 | 0.030 | 50.000 |
| | 1.00 | 1.00 | | 0.020 | 0.020 | 50.000 |
| Ethynodiol | 1.00 | 1.00 | Mestranol | 0.100 | 0.070 | 14.286 |
| diacetate | | | | | | |
| Ethynodiol | 1.00 | 1.00 | EE | 0.050 | 0.050 | 20.000 |
| diacetate | 1.00 | 1.00 | | 0.035 | 0.035 | 28.571 |
| dl-Norgestrel | 0.50 | 0.75 | EE | 0.050 | 0.050 | 10.000 |
| | 0.30 | 0.45 | | 0.030 | 0.030 | 10.000 |
| Levonorgestrel | 0.10 | 0.35 | EE | 0.020 | 0.020 | 5.000 |
| | 0.15 | 0.52 | | 0.030 | 0.030 | 5.000 |

Equivalencies
50 mg Mestranol = approx. 35 mg Ethinyl estradiol (EE)
0.1 mg dl-Norgestrel = approx. 0.15 mg Norethindrone

Each block in Table 1 describes a specific combination of progestin and estrogen, e.g., norethynodrel and mestranol, and within each block older combinations are listed first, with successively newer combinations following.

In some aspects of the invention in which an extended cycle regimen is selected, the combined estrogen and progestin is administered, e.g., for about 60 to about 110 consecutive days, or for about 80 to about 90 consecutive days. In other aspects, the period of administration is about one year, more than one year but less than two years, two years, or more than two years. In some aspects, the period of administration is continuous.

The period of administration of the combined estrogen and progestin is optionally followed by a period of about 2 to about 10 days during which neither estrogen nor progestin is administered (“hormone-free interval”). In some aspects of the invention, the hormone-free interval is about 2 to about 8 days. In other aspects, the hormone-free interval is about 2 to about 5 days. In some aspects of the invention, the hormone-free interval is achieved by administering a hormone-free placebo during that period.

In other aspects of the invention, the period of administration of the combined estrogen and progestin is optionally followed by administration of estrogen for a period of about 2 to about 10 days, (“unopposed estrogen interval”), resulting in a “bridged extended cycle regimen.” In some aspects of the invention, the unopposed estrogen interval is about 5 to about 8 consecutive days, about 3 to about 7 consecutive days, about 2 to about 7 consecutive days, or about 2 to about 5 consecutive days. In other aspects of the invention, the unopposed estrogen interval is about 7 days. In yet other aspects, it is about 3 days. See, for example, U.S. Patent Pub. No. US 2003/0139381 A1 (Bell et al.), the entire contents of which are incorporated herein by reference.

The bridged extended cycle regimen is optionally administered with an antidepressant. In some aspects of the invention, the antidepressant is administered in combination with estrogen during the unopposed estrogen interval of the bridged regimen. In other aspects of the invention, the antidepressant is administered continuously throughout the regimen, or, in yet other aspects of the invention, the antidepressant is administered intermittently. For example, in one aspect of the invention, the antidepressant is administered intermittently during the late luteal phase, which is typically one to two weeks before menses. In yet other aspects of the invention, the antidepressant is administered one time during a menstrual cycle, or once weekly.

All contraceptive regimens selected according to the method of the invention include administration to a female beginning at Day 1 of a menstrual cycle that is defined as beginning at the first day of menstrual flow. Alternatively, the selected contraceptive regimens can also include administration to a female beginning at Day 1 of a menstrual cycle that is defined as beginning with the day after the ending of the menstrual flow. Alternatively, the selected contraceptive regimens also can include administration to a female beginning at Day 1 of a menstrual cycle that is defined as beginning with any day within the menstrual cycle.

In those aspects of the invention in which the body weight of the female is less than about 150 pounds, i.e., a “lower-weight female,” a low-dose contraceptive regimen is selected.

The term “lower-weight female,” as used herein, refers to a human female who weighs less than about 150 pounds (approximately 68.0 kg).

Thus, in some aspects, the present invention provides a method of providing contraception to a lower-weight female in need thereof, the method comprising:

(a) determining a body weight of the female;

(b) designating the female as a lower-weight female if the body weight determined in (a) is less than about 150 pounds;

(c) selecting a low-dose contraceptive regimen to administer to the lower-weight female designated in (b); and

(c) administering the low-dose contraceptive regimen selected in (c) to the lower-weight female to provide contraception.

The term “low-dose contraceptive regimen” or “low dose regimen,” as used herein, refers to any contraceptive regimen that is effective in preventing or reducing the likelihood of conception, as described above, in which the daily amount of estrogen that is administered, if present, is equivalent to about 5 μg to about 30 μg of ethinyl estradiol, and in which the daily amount of progestin that is administered, if present, is equivalent to about 50 μg to about 150 μg of levonorgestrel.

In some aspects of the invention, the low-dose contraceptive regimen selected for administration comprises a combination of estrogen and progestin that is administered for a period of more than 20 consecutive days, in which the daily amount of estrogen is equivalent to about 5 μg to about 25 μg of ethinyl estradiol, and in which the daily amount of progestin is equivalent to about 50 μg to about 125 μg of levonorgestrel.

In some aspects of the invention, the daily amount of estrogen in combination with progestin in the low-dose contraceptive regimen is equivalent to about 10 μg to about 25 μg of ethinyl estradiol, or, in other aspects of the invention, is equivalent to about 10 μg to about 20 μg of ethinyl estradiol, or equivalent to about 15 μg to about 20 μg of ethinyl estradiol. In some aspects, the daily amount of estrogen in combination with progestin is equivalent to about 20 μg of ethinyl estradiol, or equivalent to about 15 μg of ethinyl estradiol.

In some aspects of the invention, the daily amount of progestin in combination with estrogen in the low-dose contraceptive regimen is equivalent to about 75 μg to about 125 μg of levonorgestrel, or, in other aspects of the invention, is equivalent to about 75 μg to about 115 μg of levonorgestrel. In some aspects, the daily amount of progestin in combination with estrogen is equivalent to about 75 μg to about 100 μg of levonorgestrel. In other aspects, the daily amount of progestin in combination with estrogen is equivalent to about 100 μg of levonorgestrel, or is equivalent to about 75 μg or levonorgestrel.

In some aspects of the invention, the daily amount of estrogen in the selected low-dose regimen is equivalent to about 20 μg of ethinyl estradiol, and the daily amount of progestin is equivalent to about 100 μg of levonorgestrel.

In some aspects of the invention, the estrogen is ethinyl estradiol, and the progestin is levonorgestrel.

The combination of estrogen and progestin, when present in the selected low-dose contraceptive regimen, is administered for a period of more than 20 consecutive days. In some aspects of the invention, the combined dosage form of estrogen and progestin is administered in an “extended cycle regimen,” i.e., the combination of estrogen and progestin is administered for a period of more than 50 consecutive days. Thus, in some aspects of the invention, the combination of estrogen and progestin is administered for a period of about 60 to about 110 consecutive days, or, alternatively, for a period of about 81 to about 89 consecutive days. In yet other aspects of the invention, the combination of estrogen and progestin is administered for a period of about one year, more than one year but less than two years, two years, or more than two years. In some aspects, the combination of estrogen and progestin is administered continuously.

Administration of the combined estrogen and progestin in the selected low-dose contraceptive regimen is monophasic, biphasic, triphasic, or multiphasic. In some aspects of the invention, the combined estrogen and progestin is administered monophasically.

In some aspects of the invention, the period of administration of the combination of estrogen and progestin in the low-dose contraceptive regimen is optionally followed by a period of about 2 to about 10 days during which neither estrogen nor progestin is administered (“hormone-free interval”), as described above. Thus, in some aspects, the hormone-free interval is about 2 to about 8 days, or about 2 to about 5 days. In some aspects of the invention, the hormone-free interval is achieved by administering a hormone-free placebo during that period.

In other aspects of the invention, the period of administration of the combination of estrogen and progestin in the selected low-dose contraceptive regimen is optionally followed by administration of estrogen for a period of about 2 to about 10 days, (“unopposed estrogen interval”), as described above (“bridged extended cycle regimen”). In other aspects of the invention, the unopposed estrogen interval is about 5 to about 8 consecutive days, about 3 to about 7 consecutive days, about 2 to about 7 consecutive days, or about 2 to about 5 consecutive days. In yet other aspects of the invention, the unopposed estrogen interval is about 7 days, or about 3 days.

In those aspects of the invention in which unopposed estrogen is administered, for example, in the bridged extended cycle regimen, the daily amount of estrogen is equivalent to about 5 μg to about 35 μg of ethinyl estradiol, or equivalent to 5 μg to about 30 μg of ethinyl estradiol, or equivalent to about 10 μg to about 30 μg, about 10 μg to about 25 μg, about 10 μg to about 20 μg, or about 15 μg to about 20 μg of ethinyl estradiol. The daily amount of unopposed estrogen administered can also be equivalent to about 30 μg of ethinyl estradiol, equivalent to about 20 μg of ethinyl estradiol, or it can be equivalent to about 15 μg of ethinyl estradiol.

The bridged extended cycle regimen, when used in a low-dose contraceptive regimen, is optionally administered with an antidepressant, as described above. Thus, in some aspects of the invention, the antidepressant is administered in combination with estrogen during the unopposed estrogen interval of the bridged regimen, i.e., in combination with the estrogen that is administered for a period of about 2 to about 10 consecutive days. In other aspects of the invention, the antidepressant is administered continuously throughout the regimen, or, in yet other aspects of the invention, the antidepressant is administered intermittently, one time during a menstrual cycle, or once weekly.

Modes of Administration and Formulations

The active agent(s) of the selected contraceptive regimen, e.g., estrogen, progestin, or a combination of estrogen and progestin, are administered in the conventional manner by any route where they are active. For example, administration can be through, but is not limited to, oral, parenteral, subcutaneous, intravenous, intramuscular, intraperitoneal, intranasal, transdermal, buccal, ocular, or vaginal routes, or by inhalation, by depot injections, or by hormone implants. Thus, modes of administration for the active agent(s) (either alone or in combination with other pharmaceuticals) can be, but are not limited to, oral, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Pharmaceutical formulations containing the contraceptive regimen active agent(s) and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder comprising an effective amount of the contraceptive regimen active agent(s) as taught in this invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, “Modern Pharmaceutics”, Banker & Rhodes, Marcel Dekker, Inc. 1979; and “Goodman & Gilman's The Pharmaceutical Basis of Therapeutics,” 6^{th} Edition, MacMillan Publishing Co., New York 1980 can be consulted.

Most contraceptive regimen active agent(s), e.g., estrogen and/or progestin, are orally active and this route of administration can be used in the invention. Accordingly, administration forms can include, but are not limited to, tablets, dragees, capsules and pills, which contain the active agent(s) and one or more suitable pharmaceutically acceptable carriers.

For oral administration, the active agent(s) of the contraceptive regimen can be formulated readily by combining these compounds with pharmaceutically acceptable carriers well known in the art. Such carriers enable the active agent(s) to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, calcium carbonate, calcium phosphate, fillers such as sugars, including, but not limited to, lactose, sucrose, mannitol, and sorbitol, cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used for oral administration include, but are not limited to, push-fit capsules' made of gelatin, as well as soft, sealed capsules made of gelatin and a plasticizer, such as glycerol or sorbitol. The push-fit capsules can contain the active agent(s) in admixture with filler such as, e.g., lactose, binders such as, e.g., starches, and/or lubricants such as, e.g., talc or magnesium stearate and, optionally, stabilizers. In soft capsules, the active agent(s) can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions of the contraceptive regimen active agent(s) can take the form of, e.g., tablets or lozenges formulated in a conventional manner.

For administration by inhalation, the contraceptive regimen active agent(s) for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The contraceptive regimen active agent(s) can be formulated for parenteral administration by injection, e.g., by bolus injection or continuous infusion. The active agent(s) can be administered by continuous infusion subcutaneously over a period of about 15 minutes to about 24 hours. Formulations for injection can be presented in unit dosage form, e.g., in ampoules or in multi-dose containers, with an added preservative. Compositions of the active agent(s) can take such forms as suspensions, solutions or emulsions in oily or aqueous vehicles, and can contain formulatory agents such as suspending, stabilizing and/or dispersing agents.

The contraceptive regimen active agent(s) can also be formulated in rectal compositions such as suppositories or retention enemas, e.g., containing conventional suppository bases such as cocoa butter or other glycerides.

In addition to the formulations described previously, the contraceptive regimen active agent(s) can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the contraceptive regimen active agent(s) can be formulated with suitable polymeric or hydrophobic materials (for example as an emulsion in an acceptable oil) or ion exchange resins, or as sparingly soluble derivatives, for example, as a sparingly soluble salt.

For transdermal administration, the contraceptive regimen active agent(s) can be applied by any transdermal, therapeutic system that is consequently supplied to the organism, such as, for example, as a transdermal patch, transdermal cream or plaster. For example, the contraceptive regimen active agent(s) can be formulated as a transdermal patch. The preparation and use of transdermal patches are well known to those of skill in the art and are available in different designs, including matrix-type or reservoir-type designs. In addition to the contraceptive regimen active agent(s), transdermal patches can contain additional components such as penetration-enhancing agents and/or additional excipients that are conventionally employed, such as, e.g., carriers, gelling agents, suspending agents, dispersing agents, preservatives, stabilizers, wetting agents, emulsifying agents, and the like.

For vaginal administration, the contraceptive regimen active agent(s) can be formulated as vaginal gels, creams, tampons, suppositories, vaginal rings, intrauterine devices and the like. The preparation of each of these formulations is well known to those of skill in the art.

The contraceptive regimen active agent(s) can also be administered according to the methods of the present invention in combination with other pharmaceutically active agents or compounds, including, for example, glucocorticoids such as vitamin D or vitamin D analogues; and/or minerals such as, for example, calcium. For example, the contraceptive regimen active agent(s) can be administered with vitamin D and/or calcium in an extended cycle regimen as a method of maintaining or preventing loss of bone density. The form of vitamin D and of calcium used in the present invention would be well known to those of skill in the art, as would the amount. For example, calcium can be administered in the form of calcium carbonate, at a daily dosage level of about 500 mg.

Examples of other pharmaceutically active agents that can be administered with contraceptive regimens according to the methods of the invention include, but are not limited to, one or more of the B complex vitamins, such as vitamin B3 (niacin (i.e., nicotinic acid and/or nicotinamide)), vitamin B9 (folic acid or folate), vitamin B6 and/or vitamin B12; iron (e.g., ferrous iron, such as, e.g., ferrous sulfate, ferrous fumarate, ferrous gluconate, or an iron glycine amino acid chelate); bisphosphonates (e.g., alendronate); teriparatide (e.g., FORTEO™); and SERMs (selective estrogen receptor modulators, e.g., raloxifene).

The contraceptive regimen active agent(s) can also be administered with an antidepressant, such as, for example, a selective serotonin reuptake inhibitor (SSRI), a tricyclic antidepressant or anxiolytic, or any antidepressant known to one of skill in the art. Suitable antidepressants include, but are not limited to, alprazolam (XANAX®), clomipramine (ANAFRANIL®), fluoxetine (PROZAC®), paroxetine (PAXIL®), sertraline (ZOLOFT®), nefazodone (SERZONE®), fenfluramine (PONDIMIN®) and venlafaxine (EFFEXOR®).

The additional pharmaceutically active agents can be administered using any suitable modes of administration, including, but not limited to, parenteral, oral, buccal, rectal, subcutaneous, intravenous, intramuscular, intranasal, transdermal modes of administration, and by inhalation. In some aspects of the invention, the additional active agent is administered using the same mode of administration as the contraceptive regimen active agent(s). For example, the additional active agent and the contraceptive regimen active agent(s) are administered together using the same mode of administration, either in the same dosage form (e.g., transdermally, using the same vaginal ring) or, alternatively, in two different dosage forms (e.g., as two separate vaginal creams). In other aspects, the additional active agent is administered using a different mode of administration, e.g., the contraceptive regimen active agent(s) are administered transdermally, using a transdermal delivery device such as a vaginal ring, and the additional active agent, e.g., an antidepressant, is administered orally, in the form of a pill or tablet.

The dosage of the additional active agent or compound can be determined readily by one of skill in the medical arts and will depend upon the condition or disorder to be treated, the physiological effect desired, and the mode of administration. For example, the amount of antidepressant administered with the contraceptive regimen active agent(s), depending on the antidepressant used, is about 0.75 to about 2 mg/24 hours, about 10 to about 20 mg/24 hours, or about 50 to about 100 mg/24 hours. Thus, in some aspects of the invention, the contraceptive regimen active agent(s) are administered with about 5 mg to about 120 mg/24 hours of fluoxetine hydrochloride. As another example, calcium administered with the contraceptive regimen active agent(s) can be in the form of calcium carbonate, at a daily dosage level of about 500 mg.

The contraceptive preparations can be produced in the form of a kit or package containing one or more dosage units to be administered according to the methods of the present invention. The kit or package can contain one dosage unit, or more than one dosage unit (i.e., multiple dosage units). If multiple dosage units are present in the kit or package, the multiple dosage units can be optionally arranged for sequential administration.

The kits of the present invention can optionally contain instructions associated with the dosage units of the kits. Such instructions can be in a form prescribed by a governmental agency regulating the manufacture, use or sale of pharmaceuticals or biological products, which notice reflects approval by the agency of the manufacture, use or sale for human administration to treat a condition or disorder. The instructions can be in any form which conveys information on the use of the dosage units in the kit according to the methods of the invention. For example, the instructions can be in the form of printed matter, or in the form of a pre-recorded media device.

“Printed matter” can be, for example, one of a book, booklet, brochure or leaflet. The printed matter can describe the use of the dosage units of the kit according to the methods of the present invention. Possible formats include, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette or audio compact disk. The information contained on the pre-recorded media device can describe the proper use of the dosage units for contraception, or for the treatment of one or more of the conditions or disorders as described herein, e.g., a menstrual disorder such as dysfunctional uterine bleeding or premenstrual symptoms.

Thus, in some aspects, the invention provides a kit containing a low-dose contraceptive preparation for providing contraception to a lower-weight female, with one or more dosage units, each dosage unit comprising estrogen, progestin, or a combination of estrogen and progestin, and with instructions for administering the one or more dosage units. The instructions indicate that the low-dose contraceptive preparation is to be administered to a lower-weight female weighing less than about 150 pounds.

Thus, the present invention thus provides a kit for providing contraception to a lower-weight female, the kit comprising:

(a) one or more dosage units, each dosage unit comprising estrogen, progestin, or a combination of estrogen and progestin; and

(b) instructions indicating that the one or more dosage units are to be administered to the lower-weight female if the lower-weight female weighs less than about 150 pounds.

In some embodiments, the kit comprises one or more dosage units comprising a low-dose contraceptive preparation comprising a combination of estrogen and progestin to be administered to the female for a specified period of time, followed by one week of non-administration. The kit also contains instructions indicating the proper use of the vaginal ring, and also indicating that the vaginal ring is to be administered to the lower-weight female if the lower-weight female weighs less than about 150 pounds.

For example, in some embodiments, the kit comprises one or more vaginal rings, each vaginal ring comprising a combination of estrogen and progestin, with each vaginal ring to be administered to the female for a period of about one week to about three months, e.g., about one week to about three weeks, about one week to two weeks, or about three weeks. For example, in some embodiments, the kit contains one vaginal ring comprising a combination of estrogen and progestin, to be administered to the female for a period from about three weeks, followed by one week of non-administration. The kit also contains instructions indicating the proper use of the vaginal ring, and also indicating that the vaginal ring is to be administered to the lower-weight female if the lower-weight female weighs less than about 150 pounds.

In other embodiments, the kit comprises two or more vaginal rings, each vaginal ring comprising a low-dose contraceptive preparation comprising a combination of estrogen and progestin, to be administered to the female for a specified period of time, e.g., for about three weeks to about three months. In addition, the kit contains one or more vaginal rings, each vaginal ring comprising a low dose of estrogen without progestin (“unopposed estrogen”), to be administered to the female for a period of about 2 to about 10 consecutive days, e.g., about 2 to 8 consecutive days, 3 to 7 consecutive days, or about 7 days. The kit also contains instructions indicating that the two vaginal rings are to be administered to the lower-weight female if the lower-weight female weighs less than about 150 pounds. For example, in some embodiments, the kit comprises five vaginal rings: four vaginal rings comprising a low-dose estrogen/progestin contraceptive preparation, each vaginal ring to be administered to the female for a period of about three weeks, in a defined sequence, and replaced with one of the three remaining estrogen/progestin vaginal rings, in the sequence indicated; and one vaginal ring comprising a low dose of estrogen (“unopposed estrogen”), to be administered to the female for a period of about 7 days. The kit also contains instructions indicating that the five vaginal rings are to be administered to the lower-weight female if the lower-weight female weighs less than about 150 pounds.

In yet other embodiments, the kit comprises one or more transdermal patches, each patch comprising a low-dose contraceptive preparation comprising a combination of estrogen and progestin, to be administered to the female for a specified period of time, e.g., about one to about 7 days; and one or more transdermal patches comprising a low dose of estrogen, to be administered to the female following administration of the last estrogen/progestin patch, each estrogen transdermal patch to be administered for a period of about 2 to about 10 days, e.g., for 2 to 8 consecutive days, 3 to 7 consecutive days, about 3 consecutive days, about 4 consecutive days, or about 7 days. The kit also contains instructions indicating that the low-dose contraceptive transdermal patches are to be administered to the lower-weight female if the lower-weight female weighs less than about 150 pounds.

In some embodiments of the invention, for example, the kit comprises more than twenty daily dosage units of a low-dose oral contraceptive preparation comprising estrogen, progestin, or a combination of estrogen and progestin, with instructions indicating that the low-dose contraceptive preparation is to be administered to a lower-weight female weighing less than about 150 pounds.

Thus, the present invention also provides a kit for providing contraception to a lower-weight female, the kit comprising:

(a) more than 20 dosage units, each dosage unit comprising estrogen, progestin, or a combination of estrogen and progestin; and

(b) instructions indicating that the more than 20 dosage units are to be administered to the lower-weight female if the lower-weight female weighs less than about 150 pounds.

In some embodiments, the invention provides a kit containing a low-dose oral contraceptive preparation for providing contraception to a lower-weight female, with the daily dosage units arranged for proper sequential administration in a synchronized, fixed sequence, wherein the sequence or arrangement of the dosage units corresponds to the stages of daily administration. Instructions are also included that explain the proper administration of the daily dosage units. The instructions also indicate that the low-dose contraceptive preparation is to be administered to a lower-weight female weighing less than about 150 pounds. The more than 20 daily dosage units comprise a low-dose oral contraceptive preparation comprising estrogen, progestin, or a combination of estrogen and progestin.

Thus the present invention provides a kit for providing contraception to a lower-weight female, the kit comprising:

(a) more than 20 oral dosage units, each oral dosage unit comprising estrogen, progestin, or a combination of estrogen and progestin; and

(b) instructions indicating that the more than 20 oral dosage units are to be administered to the lower-weight female if the lower-weight female weighs less than about 150 pounds.

In some embodiments, the kit contains 50 or more oral dosage units, e.g., 60 to 110 oral dosage units, 81 to 110 oral dosage units, or 81 to 89 oral dosage units, each oral dosage unit comprising a combination of estrogen and progestin. In some embodiments, each oral dosage unit contains estrogen in an amount equivalent to about 5 μg to about 30 μg of ethinyl estradiol, and progestin in an amount equivalent to about 10 μg to about 150 μg levonorgestrel.

For example, in some embodiments, the kit contains about 50 or more tablets for oral administration, each tablet containing a combination of estrogen and progestin and intended for ingestion on successive days, followed by about 2 to about 10 tablets, e.g., about 2 to about 8 tablets (“hormone free placebo”), each tablet containing neither estrogen nor progestin and intended for ingestion on successive days. In each tablet that contains the combination of estrogen and progestin, estrogen is present in an amount equivalent to about 5 μg to about 25 μg of ethinyl estradiol, and progestin is present in an amount equivalent to about 50 μg to about 125 μg levonorgestrel. Administration is daily for at least 50 consecutive days using tablets containing the both the estrogen and the progestin, followed by administration that is daily for about 2 to about 10 consecutive days using the hormone-free placebo tablets. For example, administration can be for 60 to 110 consecutive days, using tablets containing both estrogen and progestin, followed by administration for at least 2 to 8 consecutive days, using hormone-free placebo tablets. The kit also contains printed instructions for administering the tablets by ingesting one table per day in the sequence in which the tablets are arranged in the kit, and indicating that the low-dose contraceptive tablets are to be administered to a lower-weight female weighing less than about 150 pounds.

In alternative embodiments, the kit contains about 50 or more tablets for oral administration, e.g., 60 to 110 tablets, each tablet containing a combination of estrogen and progestin and intended for ingestion on successive days, followed by about 2 to about 10 tablets, e.g., about 2 to about 8 tablets, containing estrogen, each tablet intended for ingestion on successive days. The kit also contains printed instructions for administering the tablets by ingesting one tablet per day in the sequence in which the tablets are arranged in the kit, and indicating that the low-dose contraceptive tablets are to be administered to a lower-weight female weighing less than about 150 pounds.

For example, in some embodiments, a low-dose oral contraceptive is provided in kit form as a 91-day contraceptive regimen. For example, the kit can comprise 84 tablets, each tablet containing estrogen in an amount equivalent to about 20 μg of ethinyl estradiol, and progestin in an amount equivalent to about 100 μg levonorgestrel, intended for ingestion on successive days, followed by 7 hormone-free placebo tablets, intended for ingestion on successive days. The kit can also contain printed instructions for administering the tablets, indicating that the low-dose contraceptive tablets are to be administered to a lower-weight female weighing less than about 150 pounds.

In addition to instructions, the kit can optionally contain a planner. A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking the dosages of the kit may be difficult. Alternately, the planner can be a calendar which will provide a means to monitor when a dosage has been taken and when it has not been taken. This type of planner will be particularly useful for patients having unusual schedules for administering medication to themselves. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. The container can be large enough to accommodate each component and/or any administrative devices that may be necessary for use of the dosage units of the kit according to the methods of the present invention. However, in some cases, it may be desirable to have a smaller container which can be hidden in a patient's pocketbook, briefcase or pocket.

The present invention is also directed to a method of delivery of a contraceptive regimen according to the methods of the present invention to a patient in need thereof, the method comprising (a) registering in a computer readable medium the identity of a physician permitted to prescribe the contraceptive regimen; (b) providing the patient with counseling information concerning the risks attendant to the contraceptive regimen; (c) obtaining informed consent from the patient to receive the contraceptive regimen despite the attendant risks; (d) registering the patient in a computer readable medium after obtaining their informed consent; and (e) permitting the patient access to the contraceptive regimen.

The drug delivery methods of the present invention involve, inter alia, registering in a computer readable storage medium physicians who are qualified to prescribe the contraceptive regimen according to the methods of the present invention. Once registered in the computer readable storage medium, the physician can be eligible to prescribe a contraceptive regimen according to the methods of the invention to a patient in need thereof. Generally speaking, in order to become registered in the computer readable storage medium, the physician may be required to comply with various aspects of, for example, providing patient education and counseling. The registration of the physician in the computer readable storage medium can be achieved by providing the physician, for example, by mail, facsimile transmission, or on-line transmission, with a registration card or form, preferably together with educational materials concerning the contraceptive regimen. The physician can complete the registration card or form by providing information requested therein, and the registration card or form can be returned to the manufacturer or distributor of the contraceptive regimen, or other authorized recipient of the registration materials, for example, by mail, facsimile transmission or on-line transmission. The physician's information in the registration card or form is then entered into the computer readable storage medium. Suitable computer readable storage media which can be employed for registration of the physicians (as well as patients, as discussed below) will be apparent to one of ordinary skill in the art, once in possession of the teaching of the present application.

In the course of examination of a patient, the patient may request a contraceptive preparation and the physician may determine that administration of a contraceptive regimen according to the present invention is appropriate for the patient, or the physician may determine that the patient's condition (e.g., the patient may be suffering from a menstrual bleeding disorder) can be improved by the administration of a contraceptive regimen according to the methods of the present invention. Prior to prescribing the contraceptive regimen, the physician can counsel the patient, for example, on the various risks and benefits associated with the contraceptive regimen. The patient can be provided full disclosure of all the known and suspected risks associated with the contraceptive regimen. Such counseling can be provided verbally, as well as in written form. In some embodiments, the physician can provide the patient with literature materials on the contraceptive regimen, such as product information, educational materials, and the like.

In addition to receiving counseling on the risks attendant to administration of the contraceptive regimen according to the methods of the present invention, the methods of the invention further require the patient to fill out an informed consent form which is signed by the patient. Upon the completion of the informed consent form, the patient can be registered in a computer readable storage medium. The computer readable storage medium in which the patient is registered can be the same as, or different from, the computer readable storage medium in which the physician is registered.

The registration into one or more computer readable storage media of the physician and patient, according to the methods describe herein, provides a means to monitor and authorize access to the contraceptive regimen administered according to the methods of the present invention. Thus, the computer readable storage medium can serve to deny access to patients who fail to abide by the methods of the present invention. In some embodiments, access to the contraceptive regimen is in the form of a prescription, wherein the prescribing physician is registered in a computer readable storage medium, has provided counseling to the patient concerning the attendant risks of the contraceptive regimen, and has obtained informed consent from the patient, prior to prescribing the contraceptive regimen to the patient in need thereof according to the methods of the present invention.

The present invention is also directed to methods of educating consumers about the use of a contraceptive regimen administered according to the methods of the present invention, the method comprising distributing the contraceptive regimen with consumer information at a point of sale. In some embodiments, the distribution will occur at a point of sale having a pharmacist or healthcare provider.

As used herein, the term “consumer information” can include, but is not limited to, an English language text, non-English language text, visual image, chart, telephone recording, website, and access to a live costumer service representative. In some embodiments of the present invention, consumer information will provide directions for use of the contraceptive regimens according to the methods of the present invention, appropriate age use, indication, contraindications, appropriate dosing, warnings, telephone number of website address. In some embodiments, the method further comprises providing professional information to relevant persons in a position to answer consumer questions regarding the contraceptive regimen administered according to the methods of the present invention.

As used herein, the term “professional information” includes, but is not limited to, information concerning the contraceptive regimen when administered according to the methods of the present invention that is designed to enable a healthcare professional to answer costumer questions.

A “relevant person,” as used herein, includes, for example, a physician, physician assistant, nurse practitioner, pharmacist and customer service representative.

All of the various aspects, embodiments and options described herein can be combined in any and all variations. The contraceptive regimens that are selected for administration according to the present invention can be administered to females of child-bearing age or peri-menopausal females.

The following examples are illustrative, but not limiting, of the method and compositions of the present invention. Other suitable modifications and adaptations of the variety of conditions and parameters normally encountered and obvious to those skilled in the art are within the spirit and scope of the invention. Thus, the breadth and scope of the present invention should not be limited by any of the above-described exemplary embodiments, but should be defined only in accordance with the following claims and their equivalents.

### EXAMPLE 1

In this example, a 43-year old peri-menopausal woman consults a physician to obtain a prescription contraceptive for the purpose of preventing pregnancy. A licensed health care professional weighs the woman during the office visit and records the woman's weight as 125 pounds. The physician then examines the woman and, based upon her weight of 125 pounds, classifies her as a lower-weight woman.

The physician then selects a low-dose estrogen/progestin combination oral contraceptive regimen to prescribe to the woman, e.g., a contraceptive regimen in which the daily amount of estrogen that is administered is equivalent to about 5 μg to about 30 μg of ethinyl estradiol, and in which the daily amount of progestin that is administered is equivalent to about 10 μg to about 150 μg of levonorgestrel.

The low-dose contraceptive regimen that the physician selects is an oral contraceptive regimen in which a combination of about 20 μg of ethinyl estradiol and about 100 μg of levonorgestrel is administered daily for 84 days, followed by a “hormone-free” period of 7 days during which neither ethinyl estradiol nor levonorgestrel is administered. The low-dose oral contraceptive regimen is available in kit form, which contains 84 tablets, each tablet containing about 20 μg of ethinyl estradiol and about 100 μg of levonorgestrel, followed by 7 “hormone-free” placebo tablets, each placebo table containing neither ethinyl estradiol nor levonorgestrel. The kit also contains instructions for use, indicating that one table is to be ingested per day in the sequence in which the tablets are arranged in the kit, beginning with the first of the 84 tablets containing both ethinyl estradiol and levonorgestrel, followed by the 7 “hormone-free” placebo tablets. The instructions also indicate that the low-dose contraceptive preparation is to be administered to a lower-weight female weighing less than about 150 pounds.

The female obtains the prescribed low-dose oral contraceptive kit and administers the tablets to herself, following the instructions for use that accompany the kit.

### EXAMPLE 2

This example provides the protocol for a randomized clinical study evaluating a 91-day extended cycle oral contraceptive (OC) containing 84 days of 20 μg ethinyl estradiol (EE) and 100 μg levonorgestrel (LNG) followed by 7 days of placebo.

Materials and Methods

Study Design and Population

This is a parallel, randomized, multicenter, open-label study of a 91-day extended cycle OC (30 μg EE/150 μg LNG) and Nordette® (30 μg EE/150 μg LNG). Eligible women are randomized in a 2:1 fashion to the 91-day extended cycle OC or Nordette®. Study therapy is administered for one year (four consecutive cycles of the 91-day extended cycle regimen or 13 consecutive cycles of 28-day (conventional) cycle regimen). In the same study design, patients can also be randomized to a second 91-day extended cycle OC having a lower concentration of ethinyl estradiol (20 μg EE/100 μg LNG) or Levlite®. The intent of the study was to compare the effects of like dosage levels of the 91-day extended cycle regimen to conventional 28-day cycles within the context of separate pair-wise comparisons, and their effects with respect to body weight.

The study is performed in accordance with the Declaration of Helsinki (Republic of South Africa, 1996), applicable guidelines for Good Clinical Practice and with ethics committee approval at each participating clinical site.

Sexually active, adult women (age 18 through 40), of childbearing potential, in a heterosexual relationship, who were at risk for pregnancy, fluent in English, and able to give informed consent are eligible for the study. Active smokers >35 years old are excluded, as are women with any contraindication to the use of OCs, those who have received injectable hormone therapy (e.g., Depo-Provera®) within the 10 months prior to study enrollment, have had a progestin-releasing intrauterine device (IUD) in place within three months prior to enrollment, or a contraceptive implant removed within one month prior to enrollment. Routine use of other forms of contraception other than OCs (with the exception of condoms) is also an exclusion to study entry. Those with a recent surgical or medical abortion, miscarriage, or vaginal or cesarean delivery must have at least two normal menstrual cycles prior to enrollment. Other exclusions include history of abnormal bleeding (breakthrough or withdrawal bleeding that lasts 10 or more consecutive days, or spotting that lasts more than 10 consecutive days) while on conventional OCs, participation in any clinical investigation within 30 days prior to enrollment, and donation or sustained a loss of more than 500 mL of blood within 30 days prior to enrollment. Prohibited medications include use of any medication that might interfere with the efficacy of OCs (e.g., rifampin, barbiturates, antibiotics). At the time of entry into the study, patients are designated as continuous users (those who have been on OCs during the cycle prior to entering the study), fresh starts (those with no prior history of OC use), or prior users (those who have a history of OC use in the past without having any OC use in the 6 months prior to enrollment).

Patients can discontinue from the study for any of the following reasons: any condition that contraindicates the use of OCs, patient decision, pregnancy, any adverse event that makes continuation in the study impossible or inadvisable, lost to follow-up, discovery after enrollment not to have met study criteria, refusal to cooperate with required study procedures, or significant lapse of study medication intake (i.e., <80% overall pill taking).

Dosing Regimen and Procedures

Patients randomized to the extended cycle regimens are given blister packs with a 91-day supply of study medication (84 active pills and 7 placebo) at each clinic visit. Four pill packs are dispensed during the one-year study. Patients randomized to the conventional 28-day regimen are supplied with three or four commercial pill packs at each clinic visit, depending on the study month when the next scheduled clinic visit was to take place. All patients receive copies of patient instructions for use with each supply of study medication. They are also instructed to return all used pill packs and pill counts are conducted at each clinic visit.

Screening prior to initiation of study therapy and after obtaining informed consent include a medical and contraceptive history, physical examination (including pelvic exam and Pap smear), measurement of vital signs (including weight), clinical laboratory tests (CBC, serum chemistry, lipid profile, and urinalysis), and a urine pregnancy test. Urine pregnancy tests are also obtained at all clinic visits after baseline and at the time of study completion or patient discontinuation.

All patients enrolled in the study complete a daily electronic diary. Questions regarding pill-taking, and occurrence and severity of bleeding/spotting are recorded daily, while responses regarding concomitant contraceptive use and menstrual symptomatology are recorded weekly. All diary entries were time and date stamped to prevent retrospective completion. The diaries are programmed with a reminder alarm in the event more than 24 hours lapsed between diary entries. Patients are provided with paper diaries listing the same questions in the event of electronic diary failure or loss. Concomitant medications and adverse events are recorded separately. At each clinic visit all data from the electronic diaries are downloaded into the investigational site's study database and into a centralized database maintained by the diary vendor.

All patients are to initiate OC therapy on the first Sunday following the beginning of their menstrual period or withdrawal bleed from prior oral contraceptive cycle (“Sunday starters”) and are to remain as Sunday starters throughout the study. They are counseled to take their pill at approximately the same time each day and to record pill intake in the electronic diaries on a daily basis. There are no dosage adjustments, other than in the event that pills are missed.

Patients are seen approximately every three months during the course of the study and at the end of the study. Any patient who withdraws or who is withdrawn from the study has an end-of-study evaluation completed in the same manner as those who complete the full term of study participation. All patients are followed for two months for the occurrence of pregnancy following completion of the study or early withdrawal. Patients who become pregnant are followed for eight weeks following delivery or termination of the pregnancy. Infants are followed for eight weeks following delivery. Compliance with study medication is assessed by daily self-reporting by the patient in electronic or paper diaries. “Compliant use” is defined by eliminating all cycles in which a patient skips two or more consecutive pills, has a pattern of substantial non-compliance (<80% overall pill-taking), uses alternative forms of contraception (including emergency contraceptives), or uses prohibited concomitant medications that interact with OC therapy. “Compliant use” patients are also restricted to those patients between the ages of 18 and 35 years, according to the US Food and Drug Administration definition of “optimal” age range for ovulation (see “FDA Guidance for Industry. Combined Oral Contraceptive Labeling for Healthcare Providers and Patients,” U.S. Department of Health and Human Services, Food and Drug Administration, Center for Drug Evaluation and Research (CDER), June 2000 (http://www.fda.gov/cder/guidance/2448dft.pdf).

Efficacy Assessments

Efficacy is evaluated as the method failure rate, calculated by life table analysis and the Pearl Index (the number of pregnancies per 100 women per year of use) among women age 18-35 who use the product as directed (“compliant use”).

Pregnancy is defined by a positive urine pregnancy test and confirmed by serum human chorionic gonadotrophin (HCG). Conception date is calculated considering all available data such as sonogram data, quantitative HCG, qualitative HCG, pelvic examination, and delivery date. If the conception date is unknown, it is imputed as the midpoint between the patient's last negative pregnancy test date and the date of the positive pregnancy test. It will be assumed that the patient is “on study” at the time of conception if the pregnancy occurs between the first and last days of study drug treatment or if the conception date is completely unknown. If conception clearly occurs before the first date of taking study medication, or more than 14 days after the last dose of study drug, it will not be counted as a “during study” pregnancy.

Cycle Control Assessment

Cycle control is evaluated by observing the extent of withdrawal bleeding/spotting and breakthrough bleeding/spotting as reported in the electronic diaries. Patients are instructed that bleeding is defined as vaginal blood loss requiring the use of sanitary protection (pads or tampons). Spotting is defined as vaginal blood loss not requiring sanitary protection. All patients will respond to questions regarding the presence and intensity of bleeding and/or spotting via a series of preprogrammed questions administered on a daily basis through the electronic diaries.

Bleeding and spotting during each cycle (91 days for the extended cycle regimen and 28 days for conventional regimen), and across the full year of treatment (364 days) are evaluated by assessment of total number of bleeding and/or spotting days, number of “scheduled” bleeding and/or spotting days (i.e., occurring during the 7-day placebo pill interval), and the number of “unscheduled” bleeding and/or spotting days (i.e., occurring during the 84 day active pill interval for the extended cycle regimen or during the 21-day active pill interval for the conventional regimen). Amenorrhea is defined as a lack of withdrawal bleeding during placebo pill intervals.

Evaluation of scheduled bleeding days (i.e., that occurring during the placebo pill interval over the course of one year's treatment) is based on a total of 28 possible days for the extended cycle regimen versus 91 possible days for the conventional regimen. Evaluation of unscheduled bleeding days (i.e., that occurring during the active pill interval) is based on a total of 336 possible days for the extended cycle regimen versus 273 possible days for the conventional regimen.

Safety Assessment

Safety is evaluated by assessment of self-reported adverse events and adverse events elicited at clinic visits, clinical laboratory tests, vital signs (including weight), and physical examination. All patients who take the study drug are included in the safety assessment. A cohort of patients from the extended cycle regimen groups undergo endometrial biopsy prior to initiation of study medication, and again at the completion of the study to assess the effect of an extended oral contraceptive cycle on the endometrium. A case report form (CRF) is used to formally record all information regarding reported adverse events. Adverse events are reported spontaneously by the patients, primarily during the regularly scheduled study visit but also by way of the patient's daily diary. For each adverse event, clinical site personnel (a physician or nurse) obtain and record additional information pertaining to the seriousness of the adverse event, its severity (mild, moderate, severe, life-threatening), its onset and resolution dates, whether it is still ongoing, the action taken as a result of the event (e.g., no action taken, medical/surgical treatment, interruption of study drug, study discontinuation), and the outcome of the event on the patient's participation in the study (e.g., no effect on participation, study discontinuation, resolution with or without sequelae, death). Verbatim reported adverse events are classified by body system and preferred term using the MedDRA 4.0 coding nomenclature, a well recognized and standardized system for reporting the incidence and prevalence of adverse events in clinical trials conducted across all therapeutic areas.

Statistical Methods

For this multicenter study, data are pooled across centers. No formal statistical tests are conducted, but descriptive statistics are computed. Life table estimates of the cumulative rate of pregnancy at 52 weeks use 4-week (28-day) intervals for the conventional cycle regimen and 91-day intervals for the extended cycle regimen. Two-sided 95% percent confidence intervals are computed about each cycle's point estimate of the cumulative pregnancy rate. Since the life-table approach is based on a continuous time interval, it includes a patient's entire term of participation in the study, not just completed cycles. The Pearl Index is calculated by dividing the number of on-treatment pregnancies by the total number of complete cycles of exposure (91 days for the extended cycle regimen, and 28 days for the conventional regimen) and expressing the result as an annualized estimate per 100 subjects. The cycles of exposure also include any cycle when a pregnancy occurred.

Study Population and Disposition

The demographic characteristics of the patients in these treatment groups are identified in terms of racial distribution, mean age, body weight, body mass index, smoking status and history of OC use.

Compliance

Two measurements of compliance are evaluated by assessing patient diary data as to whether or not they took their OC pill every day. Pill compliance within each extended or conventional cycle is determined by observing if the patient misses two consecutive days of pill taking and, if so, the patient is considered to be non-compliant for that cycle. Overall study compliance is determined by counting the percentage of total days in the one-year study when the patient takes the designated pill for a given day. Overall compliance of less than 80% excludes a patient altogether from the Pearl Index calculation. Otherwise, non-compliance within a particular cycle excludes that cycle only from the Pearl Index. For the life-table calculation, only the overall compliance criterion is used to exclude “non-compliant” patients from the cumulative pregnancy rate calculation, since exclusion of individual cycles from the patient's total would lead to a non-continuous, intermittently truncated time frame.

Having now fully described this invention, it will be understood to those of ordinary skill in the art that the same can be performed within a wide and equivalent range of conditions, formulations, and other parameters without affecting the scope of the invention or any embodiment thereof.

All documents, e.g., scientific publications, patents, patent applications and patent publications recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

We claim:

1. A method of providing contraception to a female in need thereof, said method comprising:
(a) determining a body weight of said female;
(b) selecting a contraceptive regimen to administer to said female based on said body weight determined in (a); and
(c) administering said contraceptive regimen selected in (b) to said female to provide contraception.

2. A method of providing contraception to a lower-weight female in need thereof, said method comprising:
(a) determining a body weight of a female;
(b) designating said female as a lower-weight female if said body weight determined in (a) is less than about 150 pounds;
(c) selecting a low-dose contraceptive regimen to administer to said lower-weight female designated in (b); and
(d) administering said low-dose contraceptive regimen in (c) to said lower-weight female to provide contraception.

3. The method of claim 2, wherein (a) or (b) is performed by said female, or wherein wherein (c) or (d) is performed by said lower-weight female.

4. The method of claim 2, wherein (a), (b), (c), or (d) is performed by a licensed health care professional.

5. The method of claim 2, wherein said female is designated as said lower-weight female if said body weight determined in (a) is less than about 130 pounds.

6. The method of claim 5, wherein said low-dose contraceptive regimen comprises a combination of estrogen and progestin that is administered for a period of more than 20 consecutive days, in which the daily amount of estrogen is equivalent to about 5 μg to about 30 μg of ethinyl estradiol, and in which the daily amount of progestin is equivalent to about 10 μg to about 150 μg of levonorgestrel.

7. The method of claim 6, wherein said daily amount of estrogen is equivalent to about 5 μg to about 25 μg of ethinyl estradiol, and in which the daily amount of progestin is equivalent to about 75 μg to about 125 μg of levonorgestrel.

8. The method of claim 7, wherein said daily amount of estrogen is equivalent to about 10 μg to about 25 μg of ethinyl estradiol, and in which the daily amount of progestin is equivalent to about 75 μg to about 115 μg of levonorgestrel.

9. The method of claim 8, wherein said daily amount of estrogen is equivalent to about 10 μg to about 20 μg of ethinyl estradiol, and in which the daily amount of progestin is equivalent to about 75 μg to about 100 μg of levonorgestrel.

10. The method of claim 9, wherein said daily amount of estrogen is equivalent to about 20 μg of ethinyl estradiol, and in which the daily amount of progestin is equivalent to about 100 μg of levonorgestrel.

11. The method of claim 6, wherein said combination of estrogen and progestin is administered continuously.

12. The method of claim 6, wherein said combination of estrogen and progestin is administered for a period of about 20 to about 30 consecutive days.

13. The method of claim 6, wherein said combination of estrogen and progestin is administered for a period of more than 50 consecutive days.

14. The method of claim 13, wherein said combination of estrogen and progestin is administered for a period of about 50 to about 110 consecutive days.

15. The method of claim 14, wherein said contraceptive regimen further comprises a hormone-free period of about 2 to about 8 consecutive days.

16. The method of claim 13, wherein said contraceptive regimen further comprises estrogen that is administered for a period of about 2 to about 10 consecutive days, in which the daily amount of estrogen is equivalent to about 5 μg to about 30 μg of ethinyl estradiol.

17. The method of claim 16, wherein said estrogen is administered for a period of about 2 to about 8 consecutive days.

18. The method of claim 17, wherein said estrogen that is administered in combination with said progestin is ethinyl estradiol, said progestin is levonorgestrel, and said estrogen that is administered for a period of about 2 to about 8 consecutive days is ethinyl estradiol.

19. The method of claim 16, wherein an antidepressant is administered (i) in combination with said estrogen that is administered for the period of about 2 to about 10 consecutive days, (ii) intermittently, (iii) one time, or (iv) once weekly.

20. A method of providing contraception to a lower-weight female in need thereof, said method comprising:
(a) determining a body weight of a female;
(b) designating said female as a lower-weight female if said body weight determined in (a) is less than about 150 pounds; and
(c) administering monophasically to said lower-weight female a combination of estrogen and progestin for a period of more than 50 consecutive days, in which the daily amount of estrogen is equivalent to about 5 μg to about 30 μg of ethinyl estradiol, and in which the daily amount of progestin is equivalent to about 10 μg to about 150 μg of levonorgestrel,
thereby providing contraception to said lower-weight female.
